(19) United States
(12) Patent Application Publication
(10) Pub. No.: US 2025/0256054 A1
(43) Pub. Date: Aug. 14, 2025

(54) Adjustable Automated Baby Patting Device

(71) Applicant: Garegin Gary Harutyunyan, Van Nuys, CA (US)

(72) Inventor: Garegin Gary Harutyunyan, Van Nuys, CA (US)

(21) Appl. No.: 18/736,604

(22) Filed: Jun. 7, 2024

Related U.S. Application Data
(60) Provisional application No. 63/551,318, filed on Feb. 8, 2024.

(51) Int. Cl. A61M 21/02; A61H 23/00; A61H 23/02; A61M 21/00

(52) U.S. Cl. CPC A61M 21/02; A61H 23/006; A61H 23/0254; A61H 2201/0169; A61H 2201/163; A61H 2201/1676; A61H 2201/169; A61H 2201/5007; A61M 2021/0022

(57) ABSTRACT

An automated baby patting device is disclosed, comprising a patting element configured to move in a reciprocating motion. The device includes a motor housed within a casing and operatively connected to a gear, a transmission mechanism connected to the gear for converting rotational force into linear reciprocating motion, and a securing mechanism for attaching the device to an infant. The device also features a speed control mechanism operatively connected to the motor to vary the speed of the reciprocating motion, a power source housed within the casing to power the motor, a control circuit to manage the operation of the motor, a protective cover for the power source to prevent the infiltration of foreign substances, and a hinge connecting the patting element to the casing to allow its movement.

### CROSS-REFERENCE TO RELATED APPLICATIONS

This application claims priority to U.S. Provisional Application No. U.S. 63/551,318, filed on Feb. 8, 2024. The entirety of the aforementioned provisional application is hereby incorporated by reference for all purposes.

### FIELD OF INVENTION

The present invention relates generally to the field of infant care devices, specifically to automated mechanical devices designed to soothe and comfort babies through adjustable patting motions and integrated audio features.

### BACKGROUND

Current baby patting devices and methods present several limitations that have driven the need for innovative solutions in this domain. Traditional baby soothing techniques often require continuous manual effort from parents, leading to physical discomfort and fatigue. The repetitive motion of patting a baby to calm them can be strenuous, particularly during extended periods, and this manual intervention may not always be effective in providing consistent comfort to the baby.

Existing mechanical patting devices lack the versatility and customization required to cater to the varied needs of infants. Many such devices are either static or offer limited movement, failing to mimic the gentle, rhythmic motion that is often most effective in soothing a baby. Additionally, these devices frequently do not incorporate auditory elements such as white noise or lullabies, which are known to have a calming effect on infants. The absence of integrated audio features necessitates the use of separate devices, adding to the complexity and inconvenience for parents.

Moreover, the safety and comfort of current baby soothing products are often compromised. Many devices are not designed with soft, baby-friendly materials, which can lead to discomfort or even potential harm to the infant. The lack of adjustability in these products also means they may not fit securely or comfortably on babies of different sizes and ages, reducing their efficacy and practicality.

Battery life and power management are additional areas where current devices fall short. Many baby patting devices require frequent recharging or battery replacement, which can be inconvenient and disrupt the soothing process. Furthermore, the absence of protective mechanisms to prevent overheating or other hazards during use and charging poses significant safety risks.

In light of these challenges, there is a clear need for a more advanced baby patting device that addresses these limitations by providing automatic, adjustable patting motions combined with soothing audio features, all while ensuring safety, comfort, and ease of use for both babies and parents. This need has driven the development of new solutions designed to offer enhanced functionality, reliability, and convenience.

It is within this context that the present invention is provided.

### SUMMARY

The present invention relates to an automated baby patting device comprising a patting element configured to move in a reciprocating motion, a motor housed within a casing and operatively connected to a gear, a transmission mechanism connected to the gear for converting rotational force into linear reciprocating motion, a securing mechanism for attaching the device to an infant, a speed control mechanism operatively connected to the motor to vary the speed of the reciprocating motion, a power source housed within the casing, a control circuit to manage the operation of the motor, a protective cover for the power source, and a hinge connecting the patting element to the casing to allow its movement.

In some embodiments, the patting element is heart-shaped, which can provide a comforting appearance and sensation for the infant.

In some embodiments, the patting element is made from a soft material, enhancing comfort and safety for the infant during use.

In some embodiments, the securing mechanism is a belt made from flannelette material, offering a comfortable and secure attachment around the infant.

In some embodiments, the belt includes an adjustable length feature to accommodate infants of different sizes, providing versatility and extended usability of the device.

In some embodiments, the device includes a music playing speaker operatively connected to the control circuit, allowing soothing sounds to be played for the infant.

In some embodiments, the control circuit is configured to manage the operation of the music playing speaker to produce audio sounds, adding an auditory component to the soothing effect.

In some embodiments, the protective cover for the power source is designed to prevent the infiltration of liquids, ensuring the safety and longevity of the device.

In some embodiments, the power source is a polymer cell battery, which provides reliable and efficient power for the device.

In some embodiments, the device includes a battery protection mechanism configured to prevent overheating during use and charging, enhancing the safety of the device.

In some embodiments, the hinge is transparent and made from acrylic material, allowing visibility of internal components while maintaining durability.

In some embodiments, the speed control mechanism includes multiple speed settings, enabling customization of the patting motion according to the infant's needs.

In some embodiments, the casing is made from fireproof and non-toxic material, ensuring the safety and durability of the device.

In some embodiments, the device includes a wireless communication module operatively connected to the control circuit, allowing remote control of the device for added convenience.

In some embodiments, the control circuit includes a microcontroller configured to manage the operations of the motor and additional features, providing efficient and coordinated functionality.

In some embodiments, the device includes a dust-proof cover for the charging port, protecting it from dust and foreign bodies.

In some embodiments, the patting element is detachable from the hinge for easy cleaning or replacement, improving hygiene and maintenance

In some embodiments, the transmission mechanism includes a frequency conversion chip to vary the motion frequency, offering more precise control of the patting motion.

In some embodiments, the device includes vibration dampening components within the casing to reduce noise and enhance smooth operation, contributing to a more pleasant user experience.

In some embodiments, the patting element includes an internal cushioning layer for additional comfort, further ensuring the infant's comfort during use.

### BRIEF DESCRIPTION OF THE DRAWINGS

Various embodiments of the invention are disclosed in the following detailed description and accompanying drawings.

FIG. 1A illustrates an example front perspective view of the automated baby patting device.

FIG. 1B illustrates an example rear perspective view of the automated baby patting device.

FIG. 1C illustrates an example rear perspective view of the automated baby patting device with the cover of the motor assembly housing removed to reveal the internal components.

FIG. 2 illustrates an example exploded view of the components of the motor assembly for the automated baby patting device.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention.

As used herein, the term “and/or” includes any combinations of one or more of the associated listed items.

As used herein, the singular forms “a,” “an,” and “the” are intended to include the plural forms as well as the singular forms, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

When a feature or element is described as being “on” or “directly on” another feature or element, there may or may not be intervening features or elements present. Similarly, when a feature or element is described as being “connected,” “attached,” or “coupled” to another feature or element, there may or may not be intervening features or elements present. The features and elements described with respect to one embodiment can be applied to other embodiments.

The use of spatial terms, such as “under,” “below,” “lower,” “over,” “upper,” etc., is used for ease of explanation to describe the relationship between elements when the apparatus is in its proper orientation.

The term “patting element” refers to any structure or component designed to move in a reciprocating motion to soothe an infant. The patting element may be of any shape, including but not limited to, heart-shaped, oval, or rectangular, and may be made from various soft materials such as silicone, foam, or fabric-covered padding.

The term “motor” encompasses any device capable of providing rotational force, including but not limited to, direct current (DC) motors, alternating current (AC) motors, stepper motors, or servo motors. The motor may be powered by various types of power sources, such as rechargeable polymer cell batteries, disposable batteries, or any other suitable energy storage device.

The term “transmission mechanism” refers to any system or component that converts rotational motion into linear reciprocating motion. This includes but is not limited to, mechanisms utilizing gears, cams, crankshafts, or levers made from materials such as stainless steel, plastic, or composite materials.

The term “securing mechanism” is intended to cover any structure or device used to attach the patting device to an infant. This may include belts, straps, harnesses, or any other securing means made from materials such as flannelette, cotton, nylon, or elastic fabrics. The securing mechanism may incorporate adjustable features such as Velcro, buckles, or snap fasteners to accommodate different sizes.

The term “speed control mechanism” encompasses any system or component capable of varying the speed of the reciprocating motion. This may include electronic speed controllers, variable resistors, or microcontroller-based systems with pre-set speed settings or user-adjustable speed controls.

The term “control circuit” refers to any electronic system or component designed to manage the operation of the motor and other features of the device. This may include microcontrollers, printed circuit boards (PCBs), integrated circuits, or any other suitable control electronics.

The term “protective cover” is intended to cover any structure designed to shield the power source or other sensitive components from external contaminants such as liquids, dust, or debris. This may include housings, enclosures, or casings made from plastic, rubber, or other impermeable materials.

The term “hinge” refers to any pivoting or flexible connection that allows the patting element to move relative to the casing. This may include mechanical hinges, flexible joints, or pivot points made from materials such as acrylic, metal, or reinforced plastic.

In some embodiments, the device may include a wireless communication module that allows remote control via a user's phone. This module may utilize standard wireless communication protocols such as Bluetooth, Wi-Fi, Zigbee, or other suitable standards. The wireless communication module may enable functionalities such as speed adjustment, audio playback control, and monitoring of battery status through a mobile application.

### DESCRIPTION OF DRAWINGS

The present invention relates to an automated baby patting device designed to address and overcome the limitations of existing baby soothing methods and devices. Traditional methods, such as manual patting, can be physically demanding and inconsistent, leading to discomfort for parents and inadequate soothing for infants. Current mechanical devices often lack versatility, adjustability, and integrated features that enhance their efficacy and convenience.

The invention disclosed herein provides a comprehensive solution by offering an automated device that mimics the gentle, rhythmic motion of manual patting. This device is designed to deliver consistent and adjustable patting motions, thereby reducing the physical strain on parents while ensuring the baby receives effective and continuous comfort. The inclusion of an adjustable speed control mechanism allows for customization to suit the individual needs of different infants, further enhancing the device's utility.

Moreover, the invention incorporates a secure and comfortable attachment mechanism, ensuring that the device remains safely in place during use. This feature not only enhances the effectiveness of the soothing motion but also ensures the safety and comfort of the infant. The use of soft, baby-friendly materials in the construction of the device further contributes to its suitability for infant care.

In addition to the mechanical soothing motion, the invention optionally integrates auditory features, such as a music playing speaker, to provide calming sounds or white noise. This dual approach, combining tactile and auditory stimuli, offers a more holistic soothing experience for infants.

The invention is designed with safety in mind, featuring protective covers for sensitive components to prevent the infiltration of liquids and other foreign substances. The inclusion of a robust power source and a battery protection mechanism ensures reliable operation and safety during use and charging.

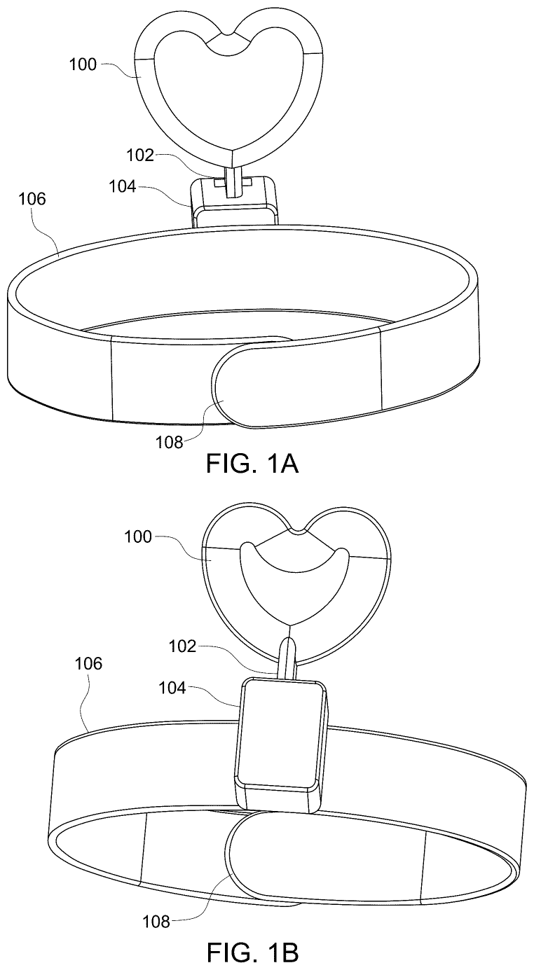

Referring now to the drawings, FIG. 1A shows a front perspective view of the automated baby patting device. The device includes a patting element 100, which is heart-shaped and designed to move in a reciprocating motion. The patting element 100 is connected to a hinge 102 that allows for its movement. The hinge 102 is attached to a casing 104, which houses the internal components. The device is secured around the infant using a belt 106, which is made from a soft flannelette material. The belt 106 features an adjustable length mechanism 108 to accommodate infants of various sizes.

FIG. 1B shows a rear perspective view of the device. The rear view highlights the casing 104, which encloses the internal components. The casing 104 also includes a protective cover for the power source, designed to prevent the infiltration of liquids and other foreign substances. The belt 106 may be secured with a Velcro fastening or the like, providing a secure and comfortable fit for the infant.

FIG. 1C shows a rear perspective view of the device with the cover of the motor 110 assembly housing removed to reveal the internal components. The internal view reveals the driving gear 114, which converts rotational force into linear motion. This driving gear 114 is connected to a transmission lever 116, which transmits the motion to the patting element 100. Additionally, the device features a music playing speaker 118 connected to the internal components, enabling the playback of soothing sounds or white noise.

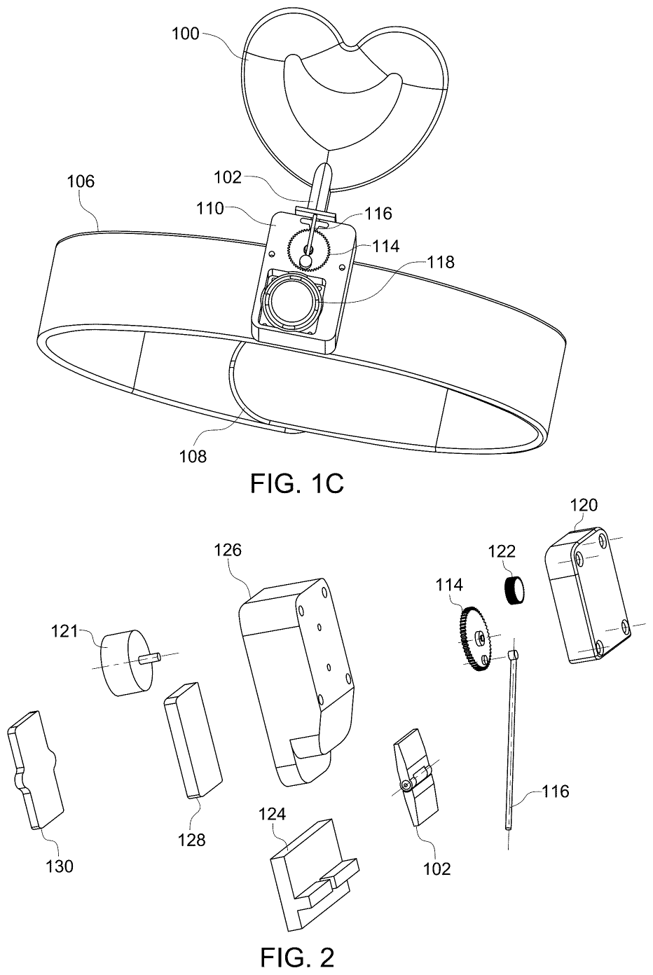

FIG. 2 shows an exploded view of the components of the motor assembly for the automated baby patting device. This view provides a detailed illustration of the internal elements and their arrangement within the casing.

The gear case 120, made from plastic, serves as the main housing for the motor assembly. Within the gear case 120 is the driving gear 122, also made from plastic. The driving gear 122 is designed to convert the rotational force provided by the electric motor 121 into linear motion. The transmission gear 114, connected to the driving gear 122, facilitates this motion conversion.

The transmission lever 116, made from stainless steel, is connected to the transmission gear 114. This lever transmits the motion to the movable plate 124, which moves in a reciprocating manner. The movable plate 124, made from plastic, is crucial for transferring the motion to the patting element (as shown in FIG. 1).

The mounting base 126, also made from plastic, supports the electric machine within the gear case 120. The transparent hinge 102, made from acrylic, allows for the movement of the patting element and is connected to the gear case 120.

The polymer cell 128, serving as the power source, is housed within the gear case 120. A protective cover 130 is included to shield the polymer cell 128, preventing the infiltration of liquids and other contaminants.

Additionally, a battery cover 132 is shown, which encloses the polymer cell 128, further ensuring its safety and integrity.

The device includes an adjustable speed control mechanism, which allows the user to vary the speed of the patting motion. This mechanism is managed by the control circuit housed within the casing. The control circuit includes a frequency conversion chip that provides precise control over the motion frequency.

In some embodiments, the device also includes a wireless communication module, allowing for remote control via a user's phone. This module utilizes standard wireless communication protocols such as Bluetooth or Wi-Fi, enabling functionalities such as speed adjustment, audio playback control, and monitoring of battery status through a mobile application.

### CONCLUSION

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the automated baby patting device of the invention have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. An automated baby patting device, comprising:
a patting element configured to move in a reciprocating motion;
a motor housed within a casing, operatively connected to a gear, said motor configured to provide rotational force;
a transmission mechanism connected to the gear, said transmission mechanism configured to convert the rotational force into a linear reciprocating motion;
a securing mechanism configured to attach the device to an infant;
a speed control mechanism operatively connected to the motor, said mechanism configured to vary the speed of the reciprocating motion;
a power source housed within the casing, configured to power the motor;
a control circuit housed within the casing, operatively connected to the motor, said control circuit configured to manage the operation of the motor;
a protective cover for the power source, configured to prevent the infiltration of foreign substances;
a hinge connecting the patting element to the casing, allowing for the movement of the patting element.

2. The automated baby patting device of claim 1, wherein the patting element is heart-shaped.

3. The automated baby patting device of claim 1, wherein the patting element is made from a soft material.

4. The automated baby patting device of claim 1, wherein the securing mechanism is a belt made from flannelette material.

5. The automated baby patting device of claim 4, wherein the belt includes an adjustable length feature to accommodate infants of different sizes.

6. The automated baby patting device of claim 1, further comprising a music playing speaker operatively connected to the control circuit.

7. The automated baby patting device of claim 6, wherein the control circuit is configured to manage the operation of the music playing speaker to produce audio sounds.

8. The automated baby patting device of claim 1, wherein the protective cover for the power source is designed to prevent the infiltration of liquids.

9. The automated baby patting device of claim 1, wherein the power source is a polymer cell battery.

10. The automated baby patting device of claim 9, further comprising a battery protection mechanism configured to prevent overheating during use and charging.

11. The automated baby patting device of claim 1, wherein the hinge is transparent and made from acrylic material.

12. The automated baby patting device of claim 1, wherein the speed control mechanism includes multiple speed settings.

13. The automated baby patting device of claim 1, wherein the casing is made from fireproof and non-toxic material.

14. The automated baby patting device of claim 1, further comprising a wireless communication module operatively connected to the control circuit, said module configured to allow remote control of the device.

15. The automated baby patting device of claim 1, wherein the control circuit includes a microcontroller configured to manage the operations of the motor and additional features.

16. The automated baby patting device of claim 1, further comprising a dust-proof cover for the charging port.

17. The automated baby patting device of claim 1, wherein the patting element is detachable from the hinge for easy cleaning or replacement.

18. The automated baby patting device of claim 1, wherein the transmission mechanism includes a frequency conversion chip to vary the motion frequency.

19. The automated baby patting device of claim 1, further comprising vibration dampening components within the casing to reduce noise and enhance smooth operation.

20. The automated baby patting device of claim 1, wherein the patting element includes an internal cushioning layer for additional comfort.
